(19) United States
(12) Patent Application Publication
(10) Pub. No.: US 2010/0221733 A1
(43) Pub. Date: Sep. 2, 2010

(54) BIOMARKER OF ALLERGIC DISEASE AND USE OF THE SAME

(75) Inventors: Tsugunobu Andoh, Toyama (JP); Yasushi Kuraishi, Toyama (JP); Tasuku Nakano, Toyama (JP)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: University of Toyama, Toyama (JP)

(21) Appl. No.: 12/713,802

(22) Filed: Feb. 26, 2010

Related U.S. Application Data
(63) Continuation of application No. PCT/JP2008/073175, filed on Dec. 19, 2008.

(30) Foreign Application Priority Data
Dec. 21, 2007 (JP) 2007-330025

(51) Int. Cl. C12Q 1/68; G01N 33/53; C12Q 1/37; C07K 16/40; C07H 21/04; C12N 15/63

(52) U.S. Cl. 435/6; 435/7.1; 435/24; 530/387.1; 536/23.2; 435/320.1; 536/24.5; 536/23.1

(57) ABSTRACT

A biomarker is provided for an allergic disease caused by an allergic reaction that is caused not exclusively by histamine release, such as pruritus, and use of the same. Use of Granzyme A as a biomarker makes it possible to provide an indication for chronic itching skin disease, for which existing antiallergic drugs have little effect, and easily and adequately allow diagnosis of the disease. It is possible to, for example, make a diagnosis of an allergic disease with an IV type allergy-like reaction not depending on the antigen-antibody reaction system. Screening with the use of Granzyme A enables the development of novel remedies for allergic diseases. Moreover, a drug capable of specifically controlling the action of a granzyme enables treatment of allergic disease with little side effect.

### CROSS-REFERENCE TO RELATED APPLICATIONS

This application is a continuation of International Application PCT/JP2008/073175, filed on Dec. 19, 2008, which designates the U.S. and which claims the benefit of Japanese Patent Application No. 2007-330025, filed on Dec. 21, 2007; both of which are hereby incorporated herein in its entirety by reference.

### BACKGROUND OF THE INVENTION

1. Field of the Invention

The present invention relates to biomarkers for allergic diseases and to the use thereof. Specifically, the present invention relates to granzyme A as a biomarker for allergic disease, such as pruritus, attributed to an allergic reaction that is not exclusively caused by histamine release, and relates to the use of granzyme A.

2. Description of Related Art

An itch is readily understood as a sensation causing a human to have a compulsion to scratch. However, since, for example, “histamine, which is a classic itch-producing substance, does not cause scratching behavior in animals such as mice” and “scratching behavior in a rat with adjuvant-induced arthritis, which is an animal model for chronic pain, is suppressed by administration of analgesics”, it is recognized that the scratching behavior of an animal is not always indicative of itching in humans.

When an itch-producing substance and a pain-producing substance are injected into the skin of the back (rostral portion near the neck) of a mouse, only the itch-producing substance causes scratching behavior by the hind paw, and the scratching behavior is indicative of itching (Non-Patent Document 1). Humans can scratch almost all portions of the body with the hand, but a mouse cannot scratch its hind paws. When itch stimulation is applied to a hind paw of a mouse, the mouse exhibits a biting reaction, and when pain stimulation is applied, the mouse exhibits a licking reaction (Non-Patent Document 2).

Most people experience strong itching when bitten by mosquitoes. It is thought that the skin reaction and the itch caused by mosquito bites are allergic reactions. First, mosquito bites barely elicit scratching behavior in mice, but scratching behavior gradually increases by repeated mosquito bites at a frequency of twice a week. The scratching behavior gradually increases even by repetitive injections of mosquito salivary gland extract. The increase in scratching behavior is also observed in mast-cell-deficient mice. In sensitized a mouse, plasma leakage from blood vessels caused by a mosquito bite is suppressed by an H₁ histamine receptor antagonist, but the scratching behavior is not suppressed. That is, in the itch due to immediate-type allergy of the skin, a mechanism other than the mast cell-histamine system is important (Non-Patent Document 3).

The scratching behavior of a mouse, which is increased by repetitive injections of mosquito salivary gland extract, is suppressed by azelastine, which is an antiallergic agent having various types of antiallergic activities, but is not suppressed by terfenadine, which is an H₁ histamine receptor antagonist, or dexamethasone, which is a steroid. Furthermore, injection of mosquito salivary gland extract into a sensitized mouse enhances the activity of primary afferent. This reaction is suppressed by azelastine, but is not suppressed by terfenadine (Non-Patent Document 4).

Non-Patent Document 1: Eur. J. Pharmacol., 275: 229-233 (1995)

### Non-Patent Document 2: Pain Res., 14: 53-59 (1999)

Non-Patent Document 3: Jpn. J. Pharmacol., 86: 97-105 (2001)
Non-Patent Document 4: J. Pharmacol. Sci., 91: 263-266 (2003)

### SUMMARY OF THE INVENTION

### 1. Technical Problem

In conventional allergy diagnosis, antigen-antibody reactions based on histamine release from mast cells have been applied in many cases. However, some reactions, such as IV-type allergy, do not depend on the antigen-antibody reaction system. In addition, known antiallergic agents have been mainly developed for stabilization of mast cell membrane and as antagonists for substances released therefrom. However, in many cases, antihistamine agents, the drugs of first choice, are ineffective for the itching of intractable pruritic skin diseases. Accordingly, development of novel antipruritic agents has been desired.

### 2. Means for Solving the Problems

The present inventors have research allergic itching by using mice sensitized with mosquito salivary gland extract, as an animal model of allergic disease such as itching, by inducing an allergic reaction with mosquito extract and, as a result, have found that (1) serine protease is released in sensitized mice by stimulation with mosquito salivary gland extract and (2) mosquito allergic pruritus is suppressed by a protease inhibitor.

In addition, the present inventors have found that no difference or only a slight difference is observed in the numbers of mast cells in sensitized mouse skin and non-sensitized mouse skin, but the number of CD4-positive (CD4⁺) T cells in the sensitized mouse skin is increased to be greater than that in the non-sensitized mouse skin.

The present inventors have further conducted intensive research and have found that protease activity was increased in the sensitized mouse skin compared to that in a non-sensitized mouse, that serine protease is released in the sensitized mice stimulated with mosquito salivary gland extract, and that the amounts of all of granzymes A, B, and C expressed in lymphocytes are increased in the sensitized mouse skin, whereas only granzyme A is expressed in CD4⁺ T cells. Furthermore, an allergic itching reaction induced by antigen stimulation and the release of granzyme A were identical in time course, and CD4⁺ T cells were not recognized in the non-sensitized mouse skin and were recognized only in the sensitized mouse skin. In addition, an itching reaction occurred in mice intradermally injected with granzyme A, and this suggested presence of an allergic reaction mediated by granzyme A. Because the release of granzyme A is specific to allergic diseases, a method for diagnosing allergy in which granzyme A is used as a biomarker was invented, and the present invention has been completed. The present invention is as follows.

[1] A biomarker for allergic disease, comprising a polynucleotide having a part of a base sequence of granzyme A gene and/or a polynucleotide complementary thereto.

[2] The biomarker according to the above [1], wherein the allergic disease is attributed to allergic reaction that is caused not exclusively by histamine release, and wherein the biomarker is used as a probe or a primer in examination of the disease.

[3] The biomarker according to the above [1] or [2], wherein the allergic disease is accompanied by pruritus.

[4] A method for diagnosing allergic disease, comprising the following steps (A) and (B):

(A) measuring the expression level of granzyme gene in a biological sample from a subject; and

(B) determining the presence or absence of the allergic disease based on the measurement result in step (A).

[5] A method for diagnosing allergic disease, including the following steps (a), (b), and (c):

(a) binding the biomarker according to any of the above [1] to [3] to an RNA prepared from a biological sample from a subject or a complementary polynucleotide transcripted from the RNA;

(b) measuring the RNA derived from the biological sample or the complementary polynucleotide transcripted from the RNA by the bound biomarker as an indication; and

(c) determining the presence or absence of the allergic disease based on the measurement result in step (b).

[6] A biomarker for an allergic disease, comprising an antibody that recognizes granzyme A.

[7] The biomarker according to the above [6], wherein the allergic disease is attributed to allergic reaction that is caused not exclusively by histamine release, and wherein the biomarker is used as an antibody for detecting granzyme A in examination of the disease.

[8] The biomarker according to the above [6] or [7], wherein the allergic disease is accompanied by pruritus.

[9] A method for diagnosing an allergic disease, including the following steps (A) and (B):

(A) measuring the expression level of granzyme A in a biological sample from a subject; and

(B) determining the presence or absence of the allergic disease based on the measurement result in step (A).

[10] A method for diagnosing an allergic disease, including the following steps (a), (b), and (c):

(a) binding the biomarker according to any of the above [6] to [8] to protein derived from a biological sample from a subject;

(b) measuring the protein derived from the biological sample by the bound biomarker as an indication; and

(c) determining the presence or absence of the allergic disease based on the measurement result in step (b).

[11] A screening method of a substance capable of suppressing expression of granzyme A, the method including the following steps (a), (b), and (c):

(a) exposing a test substance to a cell that allows measurement of the expression of granzyme A;

(b) comparing with the expression level of granzyme A in a control cell which is not exposed to the test substances by measuring the expression level of granzyme A in the cell exposed to the test substance; and

(c) selecting a test substance that can decrease the expression level of granzyme A based on the comparison result in step (b).

[12] A screening method of a substance capable of suppressing pruritus, the method including the following steps (a), (b), and (c):

(a) contacting a test substance with granzyme A;

(b) measuring inhibition of activity of granzyme A by the test substance; and

(c) selecting a test substance that can suppress pruritus based on the measurement result in step (b).

[13] The screening method according to the above [12], wherein the pruritus is due to an allergic disease.

[14] A therapeutic agent for allergic disease comprising an antibody that binds to granzyme A.

[15] A therapeutic agent for allergic disease comprising a substance that suppresses the expression level of granzyme A as an effective ingredient.

[16] The therapeutic agent according to the above [15], wherein the substance is an antibody against granzyme A or an expression vector comprising a nucleic acid molecule encoding the antibody.

[17] The therapeutic agent according to the above [15] or [16], wherein the agent has a serine protease-inhibiting activity.

[18] An allergic disease therapeutic agent comprising a substance that suppresses expression of granzyme A gene as an effective ingredient.

[19] The therapeutic agent according to the above [18], wherein the substance is an antisense nucleic acid, a ribozyme, a decoy nucleic acid, or a siRNA against granzyme A gene.

[20] The therapeutic agent according to the above [18] or [19], wherein the agent has a serine protease-inhibiting activity.

### 3. Advantageous Effects of the Invention

According to the biomarker of the present invention, it is possible to provide an index for an intractable pruritic skin disease on which conventional antiallergic agents are poorly effective, and it is possible to easily and exactly diagnose the disease. According to the method for diagnosing allergic diseases of the present invention, it is possible, for example, to diagnose allergic diseases involving reactions, such as IV-type allergy, which does not depend on antigen-antibody reactions.

In particular, the biomarker of the present invention makes it possible to identify a patient suffering from an allergic disease in which administration of antihistamine agents is ineffective or poorly effective. Under current circumstances, the antihistamine agents are prescribed as the drugs of first choice in many allergy treatments; however, if the patient is identified prior to the start of treatment and another treatment program is planned, it will make it possible to avoid unnecessary treatment, to select a more effective treatment course, to decrease economic and mental burdens on the patient, and also to reduce medical care cost.

According to the screening method of the present invention, it is possible to develop a novel allergic disease therapeutic agent based on the action mechanism of granzyme A. According to the therapeutic agent of the present invention, the activity of granzyme A can be specifically controlled, which makes it possible to treat an allergic disease with little side effects.

### BRIEF DESCRIPTION OF THE DRAWINGS

FIG. 1 is a graph showing protease activities in the skin of mice sensitized with mosquito salivary gland extract and in the skin of non-sensitized mice. The vertical axis shows relative activity of protease when the protease activity in the non-sensitized mice is assumed to be 100.

FIG. 2 is a graph showing that serine protease is released by administering mosquito salivary gland extract to mice sensitized with mosquito salivary gland extract.

FIG. 3 includes photographs for comparing the numbers of mast cells in the skin of mice sensitized with mosquito salivary gland extract and non-sensitized mice.

FIG. 4 includes photographs for comparing the numbers of CD4⁺ T cells in the skin of mice sensitized with mosquito salivary gland extract and non-sensitized mice.

FIG. 5 includes graphs showing the results of studies in RT-PCR on subtypes of granzyme mRNA expressed in the skin.

FIG. 6 is a diagram showing the results of studies of real-time PCR on subtypes of granzyme mRNA expressed in CD4⁺ T cells isolated from the skin.

FIG. 7 is a graph showing induction of scratching behavior when granzyme A is intradermally injected into normal mice.

FIG. 8 shows a graph showing that the scratching behavior induced by intradermal injection of granzyme A is suppressed by naltrexone.

FIG. 9 is a graph showing that in NC mice suffering from atopic dermatitis, the expression of granzyme A mRNA is increased in the skin of mice suffering from itching or dermatitis (conventional feeding) than that in the skin of mice not suffering from itching or dermatitis (SPF feeding).

### DETAILED DESCRIPTION OF PREFERRED EMBODIMENTS

In the present description, abbreviations for amino acids, (poly)peptides, (poly)nucleotides, and the like are in conformity with the nomenclature of the IUPAC-IUB (IUPAC-IUB Communication on Biological Nomenclature, Eur. J. Biochem., 138: 9 (1984)), “Guidelines for Preparing Specifications, etc., Containing Nucleotide Sequences or Amino Acid Sequences” (edited by the Japan Patent Office), and the conventional symbols in the relevant field.

The term “gene” or “DNA” in the present description refers to not only a double-stranded DNA but also to each single-strand of DNA constituting the double-stranded DNA, such as the sense strand and the antisense strand. Furthermore, the length is not particularly limited. Accordingly, the gene (DNA) in the present description encompasses a double-stranded DNA including a human genome DNA, a single-stranded DNA (positive strand) including a cDNA, a single-stranded DNA (complementary strand) containing a sequence complementary to the positive strand, and fragments thereof, unless otherwise specified. In addition, the “gene” or “DNA” encompasses not only a “gene” or “DNA” represented by a specific base sequence (SEQ ID NO: 1), but also a “gene” or “DNA” encoding protein (for example, homologs (including splice variants), mutants, and derivatives) having a biological function equivalent to that of the protein encoded by the above gene or DNA. Examples of the “gene” or “DNA” encoding a homolog, a mutant, or a derivative include a “gene” or “DNA” having a base sequence that hybridizes with the sequence complementary to any of specific base sequences represented by the SEQ ID NO: 1 under stringent conditions described below.

Examples of a gene encoding a homolog of a human protein include genes derived from biological species other than humans, such as mouse, rat, and the like. These genes (homologs) can be identified in HomoloGene (http://www.ncbi.nlm.nih.gov/HomoloGene/). Specifically, a human nucleic acid sequence is subjected to BLAST (Proc. Natl. Acad. Sci. USA 90: 5873-5877, 1993, http://www.ncbi.nlm.nih.gov/BLAST/) to obtain the accession number of a sequence that matches the human nucleic sequence (that is, the score is the highest, the E-value is 0, and the identity is 100%). Then, a UniGene Cluster ID (a number shown with Hs.) obtained by entering the accession number into UniGene (http://www.ncbi.nlm.nih.gov/UniGene/) is entered in HomoloGene. From the resulting list showing a gene homologous correlation between human genes and genes of living species other than humans, the genes of other species such as of mouse and rat can be selected as corresponding genes (homologs) to the human gene.

Note that the gene or DNA can contain, for example, an expression regulatory region, a coding region, an exon, or an intron, regardless of function of the regions.

In the present specification, when the term “granzyme A gene” or “DNA of granzyme A” is used, unless particularly designated by a sequence number, the human granzyme A gene (DNA) shown by a specific nucleic acid sequence (SEQ ID NO: 1) and genes (DNAs) encoding homologs, mutants, and derivative of granzyme A are included. Specifically, examples of such genes (DNAs) include human granzyme A gene (GenBank Accession No. NM_{—}006144) described in SEQ ID NO: 1 and mouse homologs thereof (for example, GenBank Accession Nos. NM_{—}010370 and XM_{—}906760).

In the present specification, the term “protein” or “(poly)peptide” refers not only to the “protein” or the “(poly)peptide” shown by a specific amino acid sequence (SEQ ID NO: 2), but also to homologs (including splice variants), mutants, derivatives, mature proteins, and amino acid-modified proteins, as long as they have biological functions equivalent to those of the original protein or (poly)peptide. Here, the homologs can be exemplified by corresponding proteins of living species other than human, such as mouse and rat, and such proteins can be identified deductively from the base sequences of the identified genes by HomoloGene (http://www.ncbi.nlm.nih.gov/HomoloGene/). The mutant includes a naturally occurring allelic mutant, an unnaturally occurring mutant, and a mutant having an amino acid sequence modified artificially by deletion, substitution, addition, or insertion. Examples of the mutant include those having at least 70%, preferably 80%, more preferably 95%, and further preferably 97% homology with the intact protein or (poly)peptide. The amino acid-modified protein includes a naturally occurring amino acid-modified body and an unnaturally occurring amino acid-modified protein. Specifically, phosphorylated amino acids are included.

In the present specification, when the term “granzyme A protein” or simply “granzyme A (hereinafter also abbreviated to GZMA)” is used, unless particularly designated by a sequence number, the terms refer to the human granzyme A shown by a specific amino acid sequence (SEQ ID NO: 2) and its homologs, mutants, derivatives, mature bodies, and amino acid-modified bodies. Specifically, the human granzyme A having the amino acid sequence described in SEQ ID NO: 2 (GenBank Accession No. NP_{—}006135.1) and mouse homologs thereof (for example, GenBank Accession No. NP_{—}034500.1) are included.

In the present specification, the term “antibody” refers to a polyclonal antibody, a monoclonal antibody, a chimeric antibody, a single-chain antibody, a humanized antibody, and their portions having antigen-binding activities, such as a Fab fragment and a fragment produced by a Fab expression library.

In the present specification, the term “biomarker” refers to those directly or indirectly used for diagnosing the presence or absence of an allergic disease, the degree of the disease, the possibility of alleviation, or the degree of alleviation, or for screening candidate substances useful for prevention or treatment of an allergic disease. Examples of the biomarker include (poly)/(oligo)nucleotides and antibodies that can specifically recognize or bind to a gene or protein for which expression varies in vivo associated with suffering from an allergic disease. The (poly)/(oligo)nucleotides and antibodies can be effectively used as probes, based on the above-described characteristics, for detecting the gene or the protein expressed in the body, tissues, or cells, and the (oligo)nucleotides can be effectively used as primers for amplifying the gene expressed in vivo.

In the present specification, the “biological tissue” subjected to diagnosis refers to tissues or cells in which the expression of a granzyme A gene is increased associated with an allergic disease caused by antigen stimulation. Specifically, skin and CD4⁺ T cells are included.

In the present specification, the term “allergic disease” includes both I-type allergy (so-called immediate-type allergy) and non-1-type allergy (non-immediate-type allergy). Preferably, the term “allergic disease” refers to an allergic disease that is attributed to immediate-type allergic reactions or non-immediate-type allergic reactions, and does not depend on histamine release alone. Allergic disease not depending on histamine release alone refers to an allergy in which a factor other than histamine such as granzyme A intermediates. That is, allergic disease accompanied by histamine release refers to diseases caused by allergic reactions mediated at least by histamine and granzyme A, and the allergic disease not accompanied by histamine release refers to diseases caused by allergic reactions mediated at least by granzyme A. Specific examples of the allergic disease include atopic dermatitis, pruritus, and itching caused by insect bites from, for example, mosquitoes, gnats, or caterpillars.

The biomarker of the present invention is characterized by including a polynucleotide containing a partial base sequence of granzyme A gene and/or a polynucleotide complementary thereof. Specifically, the biomarker of the present invention is a part of the base sequence of granzyme A gene described in SEQ ID NO: 1, for example, a polynucleotide having at least 15 consecutive bases and/or a polynucleotide complementary thereto.

Here, the term “complementary polynucleotide (complementary strand, reverse strand)” refers to a polynucleotide that is in a basically complementary relationship based on the base-pair relationship such as A:T and G:C to a partial sequence (here, for convenience, also referred to as “positive strand”), for example, a partial sequence having 15 consecutive base length, which may be from a granzyme A gene. However, the complementary strand is not limited to that forming a sequence completely complementary to the nucleic sequence of the positive strand, and may be one having a complementary relationship such that the strand can hybridize with the positive strand under stringent conditions. Note that the stringent conditions can be determined based on the melting temperature (Tm) of a nucleic acid that binds to a complex or a probe, as disclosed in Berger and Kimmel (1987, Guide to Molecular Cloning Techniques, Methods in Enzymology, Vol. 152, Academic Press, San Diego, Calif.). For example, washing conditions after hybridization are usually about “1×SSC, 0.1% SDS, 37° C.”. It is preferable that the complementary strand remains hybridized with the positive strand even after being washed under such conditions. Although not particularly limited, examples of washing conditions include about “0.5×SSC, 0.1% SDS, 42° C.” as more stringent hybridization conditions and about “0.1×SSC, 0.1% SDS, 65° C.” as further stringent hybridization conditions. Specific examples of the complementary strand include a strand composed of a nucleic acid sequence which is completely complementary to the positive strand, and a strand composed of a nucleic acid sequence having at least 90% and preferably 95% homology to the positive strand.

Here, the polynucleotide of the positive strand can be not only those including nucleic acid sequence of granzyme A gene and partial sequences thereof, but also strands composed of nucleic acid sequences complementary thereto.

Furthermore, the polynucleotide of the positive strand and the complementary strand thereof (reverse strand) may be each used as a biomarker in single-stranded form or in double-stranded form.

Specifically, the biomarker for an allergic disease of the present invention may be a polynucleotide composed of the nucleic acid sequence (full-length sequence) of granzyme A gene or the complementary sequence thereof. Furthermore, the biomarker may be a polynucleotide composed of a partial sequence of granzyme A gene or the complementary sequence thereof, as long as the polynucleotide selectively (specifically) recognizes granzyme A gene or a polynucleotide derived therefrom. In this case, examples of the partial sequence include polynucleotides having, for example, 15 consecutive nucleic acid length arbitrarily selected from the full-length sequence or the complementary sequence thereof.

Here, the term “selectively (specifically) recognize” refers to, for example, that granzyme A gene or a polynucleotide derived therefrom can be specifically detected by Northern blot, or that granzyme A gene or a polynucleotide derived therefrom is specifically amplified by RT-PCR, but this is not limited thereto as long as the detected or amplified polynucleotide can be recognized to be one derived from a granzyme A gene by one skilled in the art.

The biomarker of the present invention can be designed based on the nucleic acid sequence of human granzyme A gene described in SEQ ID NO: 1, for example, by using, for example, Primer 3 software (HYPERLINK http://www.genome.wi.mit.edu/cgi-bin/primer/primer3.cgi) or Vector NTI software (manufactured by Infomax). Specifically, a candidate sequence which can be used as a primer or a probe is obtained by applying a nucleic acid sequence of the gene of the present invention to the above software, and the obtained sequence of a part of the obtained sequence can be used as a primer or a probe.

When the biomarker of the present invention is used as a primer for detection of allergic disease, the base length is usually 15 to 100 bp, preferably 15 to 50 bp, and most preferably 15 to 35 bp. When the biomarker is used as a detection probe, the nucleotide length is usually from 15 by to the number of bases of the full-length sequence, preferably from 15 by to 1 kb, and more preferably from 100 by to 1 kb.

The biomarker of the present invention can be used as a primer or a probe according to a common procedure in a known method for specifically detecting a specific gene, such as Northern blot, RT-PCR, DNA chip analysis, or in situ hybridization. By the use of the biomarker, the presence or absence of expression or the expression level (expression amount) of granzyme A gene in an allergic disease can be evaluated.

The method for diagnosing an allergic disease of the present invention is characterized by including the following steps (A) and (B):

(A) measuring the expression level of granzyme gene in a biological sample from a subject; and

(B) determining the presence or absence of the allergic disease based on the measurement result in step (A).

For example, the method for diagnosing an allergic disease of the present invention includes the following steps (a), (b), and (c):

(a) binding the biomarker of the present invention to an RNA derived from a biological sample from a subject or a complementary polynucleotide transcripted from the RNA;

(b) measuring the RNA derived from the biological sample or the complementary polynucleotide transcripted from the RNA by the bound biomarker as an indication; and

(c) determining the presence or absence of the allergic disease based on the measurement result in step (b).

When an RNA is an object to be measured, specifically, the diagnosis method of the present invention can be practiced by, for example, Northern blot, RT-PCR, and DNA chip analysis, using the biomarker of the present invention as a primer or a probe. The increase in the amount of the biomarker-bound RNA or the transcript thereof is used as an index.

The biological sample to be measured may be total RNA extracted from biopsy samples from the subject such as a part of skin or mucous membrane, or extracted from cells collected from body fluids such as blood. The derivative nucleotide thereof may also be used as the sample to be measured. Total RNA can be prepared according to a common method.

When a Northern blot is employed, the presence or absence of expression or the expression level of granzyme A gene in RNA can be detected or measured by using the biomarker of the present invention as a probe. Specifically, the biomarker (or complementary strand) of the present invention may be labeled with, for example, a radioisotope (RI) or a fluorescent material. The labeled biomarker is subjected to hybridization with RNA derived from biological tissue of a subject transferred to a nylon membrane or the like according to a common method, and then the resulting double strand of the biomarker (DNA) and the RNA is detected or measured through the signal from the label (RI or fluorescent material) of the biomarker with a radiation detector or a fluorescence detector.

When the RT-PCR is employed, the presence or absence of expression or the expression level of granzyme A gene in RNA can be detected or measured by using the biomarker of the present invention as a primer. Specifically, cDNA may be prepared from RNA derived from a biological tissue from a subject according to a common method. PCR is performed according to a common method by hybridizing a pair of primers (a forward primer binding to the cDNA (negative strand) and a reverse primer binding to the positive strand) prepared from the biomarker of the present invention to the prepared cDNA, and amplified double-stranded DNA is detected. The amplified double-stranded DNA can be detected by labeled double-stranded DNA produced by PCR with primers previously labeled with RI or a fluorescent material, or by transferring the amplified double-stranded DNA to a nylon membrane and hybridizing with a labeled biomarker as a probe. In addition, the resulting labeled double-stranded DNA product can be measured by, for example, a bioanalyzer. Furthermore, RT-PCR reactions with SYBR Green RT-PCR Reagents (manufactured by Applied Biosystems, Inc.) can be practiced by ABI PRISM 7700 Sequence Detection System (manufactured by Applied Biosystems, Inc.).

When the DNA chip analysis is employed, a DNA chip in which the biomarker of the present invention is affixed as a DNA probe (single strand or double strand) is prepared and is subjected to hybridization with cRNA prepared from RNA derived from biological tissue of a subject by a common method, and labeled with biotin. The resulting double strand of the DNA and the cRNA is detected through fluorescence-labeled avidin.

When the in situ hybridization is employed, biological tissue of the subject is collected by biopsy, and a section is prepared. An antisense probe or a sense probe specific to the biomarker gene of the present invention is prepared. The probe is labeled with an RI label or non-RI label (for example, DIG label). The section is deparaffinized (in the case of a paraffin section), pretreated, and then fixed with ethanol or the like. The fixed section is subjected to pre-hybridization and hybridization with the probe, followed by washing and RNase treatment. The presence or absence of expression or the expression level of granzyme A gene in the biological tissue can be detected or measured by a detection method according to the label (for example, development in a case of RI labeling, and immunological detection and speculum in a case of non-RI labeling).

In the diagnosis method of the present invention, the determination of the presence or absence of the allergic disease in the step (c) is preferably conducted by comparing the measurement result of a subject with that of a normal subject and using an increase in the binding amount to the biomarker as an index.

In another aspect, the biomarker of the present invention is characterized by including an antibody that recognizes granzyme A.

The antibody is useful as a tool (biomarker) that can measure the presence or absence or the degree of an allergic disease in a subject by detecting the presence or absence or the level of granzyme A protein in a biological sample of the subject.

Furthermore, the antibody is also useful as a tool (biomarker) for detecting a variation in expression of granzyme A protein in the below-described prevention or anticipation of a symptom of an allergic disease.

The form of the antibody is not particularly limited, and the antibody may be a polyclonal antibody or a monoclonal antibody of which an immunogen is a granzyme A protein. Furthermore, the antibody may be a chimeric antibody, a single-chain antibody, a humanized antibody, or a Fab fragment produced based on a gene encoding the monoclonal antibody, or a fragment produced by a Fab expression library.

Methods for producing these antibodies are known, and the above-mentioned antibody can be produced according to a common method (Current Protocols in Molecular Biology, Sections 11.12 to 11.13 (2000)). Specifically, when the antibody of the present invention is a polyclonal antibody, the antibody can be purified from serum of an animal other than a human, such as a rabbit or a goat, immunized with granzyme A protein expressed and purified from E. coli according to a common method, or immunized with synthesized oligopeptide having partial amino acid sequence of granzyme A protein. On the other hand, a monoclonal antibody can be obtained by preparing a hybridoma. For example, such a hybridoma can be obtained by cell fusion of a myeloma cell and a spleen cell from an immunized animal other than a human, such as a mouse or a goat, with an oligopeptide having partial amino acid sequence of granzyme A protein, expressed and purified from E. coli according to a common method (Current Protocols in Molecular Biology, Edit. Ausubel, et al. (1987) Publish. John Wiley and Sons. Sections 11.4 to 11.11).

The granzyme A protein, used as an immunogen for producing an antibody, can be obtained by a process of DNA cloning based on the gene sequence information (such as SEQ ID NO: 1) of the gene provided by the present invention, construction of each plasmid, transfection into a host, culturing of the transformant, and collection of the protein from the culture. These procedures can be performed by a method known to one skilled in the art or according to a method described in a literature (for example, Molecular Cloning, T. Maniatis, et al., CSH Laboratory (1983), DNA Cloning, D M. Glover, IRL PRESS (1985)).

Specifically, a protein serving as an immunogen for producing the antibody of the present invention can be obtained by preparing a recombinant DNA (expression vector) for expressing granzyme A protein in a desired host cell, transforming a host cell by introducing the recombinant DNA, culturing the transformant, and collecting target protein from the resulting culture. Furthermore, partial peptides of the granzyme A protein also can be produced according to amino acid sequence information (for example, SEQ ID NO: 2) provided by the present invention by common chemical synthesis (peptide synthesis).

The biomarker can be included in a kit. The kit includes, for example, a polynucleotide composed of a partial base sequence of granzyme A gene and/or a complement thereof, or an anti-granzyme A antibody. When the kit includes a polynucleotide, the kit further includes, for example, dNTP, reverse transcriptase, DNA polymerase, and buffer, but is not limited thereto. One skilled in the art can select other components to be included in the kit, according to need. When the kit includes an anti-granzyme A antibody, the kit can include buffer, second antibody, marker, etc. by Western blot, ELISA, RIA, fluorescence antibody technique, or immunohistochemical staining, and one skilled in the art can select other components included in the kit according to need.

In addition, the kit can be one for directly or indirectly measuring the activity of granzyme A using an anti-granzyme A antibody and a substrate peptide.

The method for diagnosing an allergic disease of the present invention is characterized by including the following steps (A) and (B):

(A) measuring the expression level of granzyme A in a biological sample from a subject; and

(B) determining the presence or absence of the allergic disease on the basis of the measurement result in the step (A).

In one aspect, the method for diagnosing an allergic disease of the present invention is characterized by using the biomarker of the present invention. The biomarker has a property of specific binding to granzyme A protein and thereby can specifically detect the granzyme A protein expressed in tissue of an animal.

For example, the method for diagnosing an allergic disease of the present invention can be conducted by a method including the following steps (a), (b), and (c):

(a) binding the biomarker (antibody) of the present invention to protein derived from a biological sample from a subject;

(b) measuring the protein derived from the biological sample by the bound biomarker as an indication; and

(c) determining the presence of the allergic disease based on the measurement result in the step (b).

When the object to be measured is a protein, specifically, the diagnosis method of the present invention can be practiced by using the biomarker (antibody) of the present invention as the antibody for detecting granzyme A, and performing a detection method such as Western blotting, RIA, ELISA, fluorescence antibody technique, or immunohistochemical staining, and detecting the increase of the bound biomarker as an indication.

The sample to be measured may be taken by, for example, biopsy of part of tissue such as skin of the subject, protein prepared according to a common method from a sample obtained by collecting cells present in body fluid such as blood, or protein being dissolved in body fluid, according to the type of the detection method employed.

More specifically, the method diagnosing allergic diseases of the present invention can be practiced by Western blotting using the biomarker (antibody) of the present invention, and detecting the increase of granzyme A bound to the biomarker as an indication.

When Western blotting is employed, the method can be conducted by using the biomarker of the present invention as the primary antibody, then using a secondary antibody (antibody binding to the primary antibody) labeled with, for example, isotope such as ¹²⁵I, a fluorescent material, or an enzyme such as horse radish peroxidase (HRP) as the secondary antibody. The signal from the isotope or the fluorescent material can be detected by a radiation counter, a fluorescence detector, or the like.

When the immunohistochemical staining is employed, for example, a cell expressing granzyme A can be detected by using an enzyme-labeled antibody and a chromogenic substrate thereof.

The diagnosis of an allergic disease can be performed by measuring the expression level of granzyme A gene, or the amount, function, or activity (hereinafter, these may be collectively referred to as “protein level”) of granzyme A protein in, for example, skin biopsy tissue, blood, or CD4⁺ T cells of a subject.

The screening method of substances capable of suppressing expression of granzyme A of the present invention includes the following steps (a), (b), and (c):

(a) exposing test substance to a cell that allows measurement of the expression of granzyme A;

(b) comparing with the expression level of granzyme A in a control cell which is not exposed to the test substance by measuring the expression level of granzyme in the cell exposed to any of the test substances; and

(c) selecting a test substance that can decrease the expression level of granzyme A based on the comparison result in step (b).

In step (a), the test substances may be any known substance or novel substances, and examples thereof include nucleic acids, saccharides, lipids, protein, peptides, organic low-molecular compounds, compound libraries produced by using combinatorial chemistry technology, and random peptide libraries produced by solid-phase synthesis or a phage-display system, and also include natural substances derived from, for example, microorganisms, animals, plants, and marine organisms.

In step (a), examples of the cells that allow measurement of expression of granzyme A include general cultured cells that express endogenous and/or exogenous granzyme A, or cells containing reporter genes. The expression of the gene in the cultured cells can be easily confirmed by detecting gene expression by Northern blot or RT-PCR.

Specific examples of the cells include CD4⁺ T cells isolated and prepared from an animal suffering from an allergic disease or their cell strains; cells into which have been introduced with any of the genes of the present invention; and cells into which have been introduced a reporter (such as luciferase and GFP) gene.

As the animal model, any animal model that is well-known as an allergic disease animal model can be used, and examples thereof include NC-strain mice fed under normal circumstances.

Examples of the cells for gene transfection include CHO, MCF-7 mammary carcinoma cells, and H295R adrenal cells.

In step (a), the cells that allow measurement of expression of granzyme A are exposed to the test substances in a culturing medium. As the cells, CD4⁺ T cells isolated and prepared from an animal suffering from an allergic disease accompanied by pruritus can be preferably used. The culturing medium is appropriately selected according to the cells that allow measurement of expression of granzyme A, and examples thereof include minimum essential medium (MEM) containing about 5 to 20% fetal bovine serum and Dulbecco's modified minimum essential medium (DMEM), RPMI 1640 medium, and 199 medium. Similarly, the culturing conditions are appropriately determined, and for example, the medium pH is about 6 to 8, the culturing temperature is usually about 30 to 40° C., and the culturing time is about 12 to 72 hours.

In step (b), the measurement of the expression level is practiced by using mRNA or protein as the object. The expression level of the mRNA is measured by, for example, RT-PCR or Northern blot, by preparing total RNA from cells. The expression level of the protein can be measured by, for example, an immunological method by preparing extracts from cells. Examples of the immunological method include Western blotting, radioimmunoassay (RIA), ELISA, and fluorescence antibody technique. When the cell containing a reporter gene (for example, a cell into which have been introduced a vector linked with a reporter gene (for example, luciferase or GFP) on the downstream side of the promoter of granzyme A so as to be functional) is used, the expression level is measured on the basis of the signal strength of the reporter gene.

In step (b), the comparison of the expression levels is performed based on a significant difference in the expression levels of granzyme A between in the presence and the absence of a test substance. Furthermore, the expression level of granzyme A in control cells not having been exposed to the test substance may be the expression level of granzyme A previously measured or simultaneously measured with respect to the expression level measurement for the cells having been in contact with any of the test substances, but the expression level simultaneously measured is preferred from the viewpoints of accuracy and reproducibility.

In step (c), test substances that decrease the expression level of granzyme A are selected. In the thus selected test substances, those that can vary the expression level of granzyme A are included, due to the characteristics of the screening method. The selected test substances are not only candidates as therapeutic agents for allergic diseases accompanied by pruritus (for example, a therapeutic agent for atopic dermatitis, in particular, a therapeutic agent for histamine-resistant pruritus), but are also useful as reagents for research.

Furthermore, the screening method for substances capable of suppressing pruritus of the present invention includes the following steps (a), (b), and (c):

(a) bringing test substances into contact with granzyme A;

(b) measuring the activity of granzyme A inhibited by the test substances; and

(c) selecting a test substance that suppresses pruritus on the basis of the measurement result in step (b).

In the step (a), the test substances are as described above.

In the step (a), the test substances are contacted with granzyme A, and this step may be practiced according to a common process for measuring enzyme inhibition.

In step (b), the measurement of inhibition of the activity of granzyme A is conducted by measuring the amount of free nitroanilide, which has a specific absorption wavelength and is cleaved and released by the enzyme from a specific substrate peptide to which the nitroanilide is bound.

In the step (c), test substances that inhibit the activity of granzyme A are selected. The thus selected test substances are not only candidates as therapeutic agents for allergic diseases accompanied by pruritus (for example, a therapeutic agent for atopic dermatitis, in particular, a therapeutic agent for histamine-resistant pruritus), but are also useful as reagents for research.

Among the biomarkers of the present invention, a biomarker comprising the above-described antibody can prevent or treat variations in allergic disease conditions by being applied to an animal, and the present invention provides such a method. Among the above-mentioned antibodies, a neutralizing antibody is particularly preferred.

The animal is preferably human or a vertebrate animal other than a human, and is particularly preferably a domestic animal or a pet animal, such as cow, horse, pig, sheep, goat, chicken, dog, or cat.

The present invention provides an allergic disease therapeutic agent containing a substance that inhibits the level of activity of the biomarker as an effective ingredient.

The therapeutic agent of the present invention is not particularly limited as long as it suppresses the level or activity of granzyme A, and examples thereof include the above-described various types of antibodies and known serine protease inhibitors.

The therapeutic agent of the present invention is used as a therapeutic agent for an allergic disease, preferably, as a therapeutic agent for an allergic disease accompanied by pruritus.

The therapeutic agent of the present invention may be an effective ingredient itself, or may contain, for example, a known pharmaceutically acceptable carrier. Examples of the carrier include: fillers such as sucrose, starch, mannitol, sorbitol, lactose, glucose, cellulose, talc, calcium phosphate, and calcium carbonate; binders such as cellulose, methylcellulose, hydroxypropylcellulose, polypropyl pyrrolidone, gelatin, gum arabic, polyethylene glycol, sucrose, and starch; disintegrators such as starch, carboxymethylcellulose, hydroxypropyl starch, sodium carboxymethyl starch, sodium hydrogen carbonate, calcium phosphate, and calcium citrate; lubricants such as magnesium stearate, aerosil, talc, and sodium lauryl sulfate; flavors such as citric acid, menthol, a glycyl lysine ammonium salt, glycine, and orange powder; preservatives such as sodium benzoate, sodium bisulfate, methylparaben, and propylparaben; stabilizers such as citric acid, sodium citrate, and acetic acid; suspending agents such as methylcellulose, polyvinyl pyrrolidone, and aluminum stearate; dispersing agents such as surfactants; diluents such as water and physiological saline; and base wax such as cacao butter, polyethylene glycol, and paraffin; but these are not limited thereto.

As another aspect, the therapeutic agent of the present invention contains a substance that suppresses expression of the granzyme A gene as an effective ingredient.

The substance is preferably an antisense nucleic acid, a ribozyme, a decoy nucleic acid, or a siRNA against the granzyme A gene.

The term “antisense nucleic acid” refers to a nucleic acid that consists of a nucleic acid sequence capable of hybridizing with target mRNA (primary transcription product) in cells that express the target mRNA (primary transcription product) under the physiological conditions, and can inhibit the translation into a polypeptide encoded by the target mRNA (primary transcription product) under the hybridized state. The kind of the antisense nucleic acid may be DNA or RNA and may be DNA/RNA chimera.

The term “ribozyme” refers to an RNA having an enzyme activity to cleave an oligonucleotide. Since it has recently been shown that an oligo DNA having a base sequence of the enzyme activity site also possesses a nucleic acid cleavage activity, the term ribozyme is used in the present invention as a concept encompassing DNA, as long as it possesses a sequence-specific nucleic acid cleavage activity.

The term “decoy nucleic acid” refers to a nucleic acid molecule that mimics the region to be bound with a transcription regulating factor, and the decoy nucleic acid serving as a substance that inhibits expression of granzyme A can be a nucleic acid molecule that mimics the region to be bound with a transcription activating factor for granzyme A. The decoy nucleic acid in the present invention comprises oligonucleotides that are modified so as to be resistant to degradation in vivo, such as an oligonucleotide (S-oligo) having a thiophosphate diester bond in which the oxygen atom of a phosphate diester bond moiety is substituted by a sulfur atom, or an oligonucleotide in which a phosphate diester bond is substituted by a methyl phosphate group not having a charge. The decoy nucleic acid may be completely identical to the region to be bound with a transcription activating factor or may be not completely identical as long as it maintains identity at a degree such that the transcription activating factor for granzyme A can bind. The length of the decoy nucleic acid is not particularly limited as long as the transcription activating factor can bind. Furthermore, the decoy nucleic acid may repeatedly contain the regions.

The term “siRNA” refers to a double-stranded oligo RNA that is complementary to a partial sequence of mRNA or primary transcription product-coding region (in the case of the primary transcription product, the partial sequence includes an intron region) of granzyme A. By introducing a siRNA into a cell, a phenomenon, so-called RNA interference (RNAi), occurs, and the same effect as that of the ribozyme can be expected.

The above antisense nucleic acid, ribozyme, decoy nucleic acid, and siRNA can be produced according to known methods.

### EXAMPLES

The present invention will be described in more detail with reference to examples below, but is not at all limited to these examples.

### Feeding of Mosquito

The mosquitoes used in examples were adult female Aedes albopictus, kindly provided by the Department of Clinical Infectious Diseases, Faculty of Medicine, University of Toyama. The adult mosquitoes were fed in a fabric cage (30×30×30 cm) and were allowed to freely take a 3% sucrose aqueous solution. Larval mosquitoes were fed in a plastic cage (25×35×12 cm) containing ion-exchanged water while circulating air with an air pump by giving a mixture of dried yeast and baby food at a ratio of 1:1 as feed. Pupal mosquitoes were collected in a container containing ion-exchanged water and were incubated in a fabric cage.

### Preparation of Mosquito Salivary Gland Extract (ESGM) and Production of Sensitized Mice

The adult female Aedes albopictus mosquitoes were frozen, and the thorax sections were isolated by removing limbs, wings, heads, and abdominal portions under a microscope, and collected in an eppendorf tube. A small amount of distilled water was added in the tube, followed by homogenization (1500 rpm, 4° C., 5 min). After centrifugation (about 9000×g, 30 min), the supernatant was filtered through a filter (cellulose acetate: 0.45 μm, Advantec MFS, Inc.). The amount of protein was determined from absorbance at 590 nm using a Bio-Rad Dye Reagent and lyophilized bovine serum albumin (Bio-Red Laboratories, USA), and the protein was dispensed at 100 μg per eppendorf tube, followed by lyophilization and then stored at −80° C.

The ESGM was dissolved in physiological saline such that the protein amount in 50 μL was 10 μg. The resulting ESGM solution was intradermally injected into the caudal back of mice twice a week, eight times in total, for forming sensitized mice. After the sensitization, ESGM (10 μg/site) was intradermally injected into the rostral back of the mice to induce scratching behavior.

### Measurement of Tryptase-Like Serine Protease Activity in Skin

Skin tryptase-like serine protease activity was measured using a synthetic substrate of tryptase with reference to the method of Wolter, et al., (2001). N-p-Tosyl-Gly-Pro-Arg-p-nitroanilide acetate salt (Sigma Aldrich Corp.) was used as the synthetic substrate. The enzyme activity was determined by measuring the amount of free nitroanilide with a spectrophotometer (ImmunoMini NJ-2300).

On the day before the experiment, the hair on the rostral back of each mouse was removed, and on the day of the experiment, the skin (diameter: 17 mm) was sampled. The sampled skin was homogenized in 1.5 mL of a 10 mM tris solution (pH: 6.1, containing 2 M of sodium chloride). After sonication for 10 minutes and then centrifugation at 5000 rpm for 5 minutes at 4° C., the supernatant was collected. A 0.06 M tris solution (pH: 7.8, containing 0.4% dimethyl sulfoxide and 30 μg/mL of heparin) was used as a reaction solution A. A dimethyl sulfoxide solution containing 10 mg/mL of a synthetic substrate was prepared, and the solution was further diluted with the reaction solution A to prepare a 480 μg/mL substrate solution. The reaction solution (49 μL), the sample (1 μL), and the substrate solution (50 μL) were subjected to a reaction at 37° C. for 1 hour, followed by measurement of absorbance at 420 nm.

### Measurement of Free Tryptase-Like Serine Protease Activity in Skin

On the day two days before the experiment, the hair on the back of each mouse was removed. On the day of the experiment, the mouse was anesthetized with urethane (1.8 g/kg) and was fixed ventrally on a heated plate. Then, two 23 G injection needles were inserted through the skin on the rostral back of the mouse at an interval of about 1 cm. A dialytic tube through which stainless steel wire passed was inserted into the needles in the skin, and only the needles were removed without damaging the dialytic tube. A vinyl chloride tube (p-10 tube) was bonded to the dialytic tube with an adhesive, taking care not to allow the dialytic tube to dry out. Then, a solution for an enzyme (pH: 7.3, containing 0.5 mM of tris and 0.1 M of sodium chloride) was perfused in the skin at a flow rate of 1 μL/min for 1 hour using an EICOM EP-60 micro syringe pump (Eicom Corp.). This was performed as pretreatment, and then the skin perfusion solution was collected every 5 minutes in an ice bath. Samples were collected over 15 minutes after the pretreatment. The perfusion was stopped once, and 50 μL of ESGM or physiological saline was intradermally injected four times at a predetermined position near the dialytic tube without damaging the dialytic tube. Immediately after the injection, the perfusion was restarted, and the skin perfusion solution was collected over 40 minutes after the injection. An amount of 30 μL of a reaction solution B (pH 7.77, containing 85.74 mM of tris, 0.572% dimethyl sulfoxide, and 42.87 μg/mL of heparin), 50 μL of a 480 μg/mL substrate solution prepared by diluting a dimethyl sulfoxide solution containing 10 mg/mL of a synthetic substrate with the reaction solution B, and 20 μL of a sample collected from each of two mice were subjected to a reaction at 37° C. for 2 days, followed by measurement of absorbance at 420 nm.

### Isolation of CD4⁺ T Cells

On the day before the experiment, the hair on the rostral back of each mouse was removed. On the day of the experiment, the mouse was anesthetized with ethyl carbonate (1.8 g/kg) and was subjected to blood removal using a 0.1 M phosphate buffer saline solution (PBS, pH: 7.4), and the skin sterilized with ethanol was extracted. The extracted skin was immersed in 10 mL of RPMI 1640 (containing 0.25% collagenase A), followed by stirring at 37° C. for 30 minutes. Ethylenediamine tetraacetate (EDTA) was added thereto in a final concentration of 10 mM, and the resulting mixture was immediately cooled in an ice bath. After 5 minutes, floating cells were collected, and the remaining skin was washed with PBS (containing 10 mM EDTA) twice. The solution used for the washing was collected. The collected solution was applied to 70-μm and 40-μm nylon meshes, and the solution that passed through the nylon meshes was centrifuged at 2000 rpm for 20 minutes at 4° C. The supernatant was removed, followed by resuspension in 5 mL of RPMI 1640. Lymphocytes were isolated from the suspension using Lympholyte-M (Cedarlane Laboratories Ltd., Canada), and CD4⁺ T cells were continuously isolated using a CD4 column (RD systems, USA).

### Section Preparation

Each mouse anesthetized with ethyl carbonate (1.8 g/kg) was subjected to blood removal using a 0.1 M phosphate buffer saline solution (PBS, pH: 7.4) and was fixed with 4% paraformaldehyde (PFA). The skin was extracted and then fixed with 4% PFA after 4 hours, followed by substitution by 30% sucrose (containing a 0.1 M phosphate buffer solution). After 24 hours, the skin was embedded in an OTC compound (Sakura Fine Technical Co., Ltd.), followed by being frozen. Samples for toluidine blue staining were sliced with a cryostat to 20 μm and placed on a slide glass coated with gelatin, followed by being stored at −80° C. under light shielding until being stained. Samples for immunostaining were sliced with a cryostat to 40 μm and were stored in a 0.1 M PBS (containing 0.02% sodium azide) at 4° C. under light shielding until being stained.

Toluidine Blue Staining

The section was immersed in 0.1% toluidine blue for 15 to 20 minutes. When the tissue was stained blue, the section was washed with water and sealed with polymount. The tissue specimen was observed with an optical microscope (AX80, Olympus Corp.).

Immunostaining

The section was blocked with 1.5% fetal bovine serum (FCS) and then subjected to a reaction with a rat anti-mouse CD4 monoclonal antibody (1:100, BD Pharmingen, USA) at 4° C. overnight. The section was washed with PBST (PBS containing 0.2% Tween 20) twice and then subjected to a reaction with a Cy3-labeled anti-rat IgG polyclonal antibody (1:1000, Chemicon, USA) at room temperature for 2 hours. After washing with PBST, the section was placed on a slide glass, dried, and then sealed with DABCO (1,4-diazabicyclo[2,2,2] octane). The tissue specimen was observed with a confocal/multiphoton laser scanning microscope (Radeance 2100 MP; Bio-Rad Laboratories, USA).

### RNA Extraction

Each mouse anesthetized with ethyl carbonate (1.8 g/kg) was subjected to blood removal using a 0.1 M phosphate buffer saline solution (PBS, pH: 7.4). The skin and spleen were extracted and sectioned. Each sample was frozen in liquid nitrogen and stored at −80° C. until use. The sample was put in Trizol reagent (Invitrogen Corp.), followed by homogenization. After being left standing at room temperature for 5 minutes, chloroform/isoamyl alcohol (CIA) was added thereto, followed by stirring for 15 seconds and then centrifugation at 14000 rpm at 4° C. for 15 minutes. The supernatant was collected, and 100% ethanol in the same amount was added thereto, followed by stirring. The resulting mixture was put in a column (Sigma Aldrich Corp.) and centrifuged at 15000 rpm for 15 seconds to make the genetic material adhere to the column. The column was washed with wash solution I and then subjected to DNase I treatment (room temperature, 15 min) and washed with wash solution I again and further washed with wash solution II twice. Then, RNA was eluted from the column with an elution solution. The RNA level was measured using NanoDrop (LMS Corp.).

### Reverse Transcription Reaction

Sample RNA (1 μg) and oligo dT16 primer (25 μmol) were put in a PCR tube, and the total volume was adjusted to 5 μL with RNase-free water, followed by a reaction at 70° C. for 5 minutes and then quenching at 4° C. for 5 minutes. A reaction solution (15 μL) having the following composition A was added to each sample, followed by reactions at 25° C. for 5 minutes, at 37° C. for 1 hour, and 72° C. for 15 minutes in turn.

Composition A (total volume: 15 μL)

5× Reaction buffer (Wako Pure Chemical Industries, Ltd.): 4 μL

MgCl₂ (25 mM) (Wako Pure Chemical Industries, Ltd.) in a final concentration of 3 mM: 2.4 μL

dNTP (2 mM each dNTP) (ABI, USA) in a final concentration of 0.5 mM: 5 μL

RNase inhibitor (Toyobo Co., Ltd.) in a final concentration of 1 U/μL: 0.5 μL

ReverScript III (Wako Pure Chemical Industries, Ltd.): 1 μL

RNase-free water: 2.1 μL

### PCR Reaction

The reverse transcription (RT) product was mixed with the following composition B, followed by a reaction in a thermal cycler (Takara Bio Inc.). The mixture solution was subjected to a reaction at 95° C. for 2 minutes and then a reaction cycle of at 95° C. for 30 seconds, at 60° C. for 30 seconds, and at 72° C. for 50 seconds. The reaction cycle was repeated 30 times. Finally, a reaction at 72° C. was conducted for 5 minutes, and the reaction was terminated at 4° C. The PCR product was separated by electrophoresis on 1% agarose gel, and then the agarose gel was stained in ethidium bromide solution. After 20 minutes, the gel was photographed by being irradiated with UV.

Composition B (total volume: 50 μL)

5× Green GoTaq Flexi buffer (Promega, USA): 10 μL

MgCl₂ (25 mM) (Wako Pure Chemical Industries, Ltd.) in a final concentration of 1.5 mM: 3 μL

dNTP (2 mM each dNTP) (ABI, USA) in a final concentration of 0.2 mM: 5 μL

Sens primer*¹ (Hokkaido System Science Co., Ltd.) in a final concentration of 1 μM: 1 μL

Anti-sens primer*¹ (Hokkaido System Science Co., Ltd.) in a final concentration of 1 μM: 1 μL

GoTaq DNA polymerase (5 u/μL) (Promega, USA) in a final concentration of 1.25 u: 0.25 μL

Template DNA: 1 μL

Sterilized water: 28.75 μL *1: The Sens primer and the Anti-sens primer are shown in Table 1.

### Real-Time PCR

The RT product was mixed with the following composition C, followed by a reaction with Mx3000P & Mx3005P Real-Time PCR System (Stratagene, USA). The mixture solution was subjected to a reaction at 95° C. for 1 minute and then a reaction cycle of at 95° C. for 30 seconds, at 60° C. for 30 seconds, and at 72° C. for 50 seconds. The reaction cycle was repeated 40 times. Finally, a reaction at 72° C. was conducted for 5 minutes, and the reaction was terminated at 4° C. The amplification of the PCR product was analyzed by MxPro QPCR Software (Stratagene, USA).

Composition C (total volume: 25 μL)

2×SYBR Premix Ex Taq (Takara Bio Inc.): 12.5 μL

Sens primer*¹ (Hokkaido System Science Co., Ltd.) in a final concentration of 1 μM: 1 μL

Anti-sens primer*¹ (Hokkaido System Science Co., Ltd.) in a final concentration of 1 μM: 1 μL

GoTaq DNA polymerase (5 u/μL) (Promega, USA) in a final concentration of 1.25 u: 0.25 μL

Template DNA: 1 μL

Sterilized water: 10.5 μL *1: The Sens primer and the Anti-sens primer are shown in Table 1.

TABLE 1

| | | |
|---|---|---|
| name | Sens primer sequence | Anti-sens primer sequence |

| | | |
|---|---|---|
| GAPDH | 5′-CCAAGGTCATCCATGACAAC-3′ | 5′-TTACTCCTTGGAGGCCACGT-3′ |
| Granzyme A | 5′-TGGAGGAGACACGGTTGTTC-3′ | 5′-GAGGGAGCTGACTTATTGCC-3′ |
| Granzyme B | 5′-CCTACATGGCCTTACTTTCG-3′ | 5′-AACCTCTTGTAGCGTGTTTG-3′ |
| Granzyme C | 5′-AGATAATCGGAGGCAATGAG-3′ | 5′-CACCTGATCCTTCTGTACTG-3′ |

### Behavior Experiment

On the day before the experiment, the hair on the rostral back of each mouse was removed. On the day of the experiment, the mouse was left in an observation cage for 1 hour to familiarize the mouse with the environment. After the familiarization, granzyme A (0.1 to 100 μg/site) or ESGM (10 μg/50 μL) was intradermally injected to the rostral back of the mouse. The mouse was returned to the observation cage (13×9×30 cm) immediately after the administration, and the behavior with no observer present was filmed with an 8-mm video camera. A protease inhibitor in an amount of 0.05 mL per 10 g of body weight of the mouse was injected into the tail vein 30 seconds before the administration of ESGM. Naltrexone in an amount of 0.1 mL per 10 g of body weight of the mouse was intradermally injected 15 minutes before the administration of granzyme A. The behavior after the injection was observed by playing back the video. Scratching behavior was determined by counting the number of times there was scratching of the injection portion and nearly areas with the hind paws. A mouse usually scratches several times for about 1 second, and a series of this behavior was determined as one instance of scratching behavior.

### Results

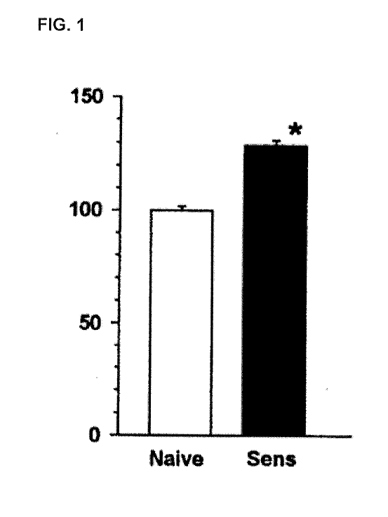

(1) The protease activity in the skin of mice sensitized with mosquito salivary gland extract is significantly increased compared to that in non-sensitized mice (FIG. 1).

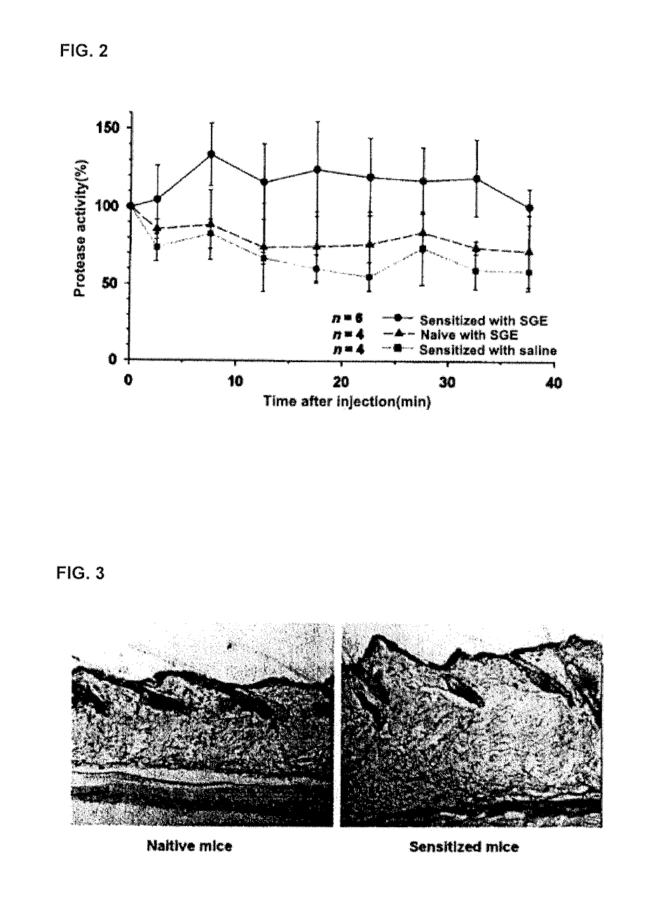

(2) In the result of an activation test using a serine protease specific substrate and a perfusion solution obtained by a skin perfusion method, serine protease is released by administering the mosquito salivary gland extract to mice sensitized with the mosquito salivary gland extract (FIG. 2).

(3) There is no difference in the numbers of mast cells in the skin of mice sensitized with mosquito salivary gland extract and in the skin of non-sensitized mice (FIG. 3).

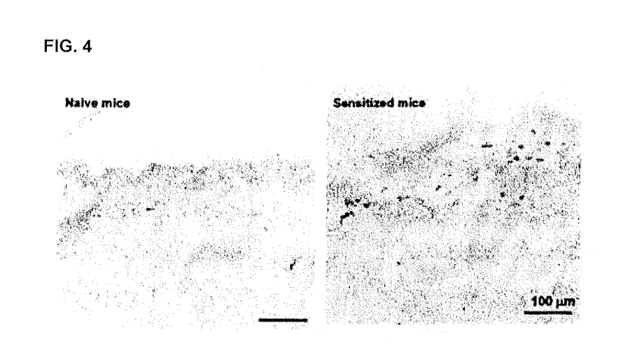

(4) The number of CD4⁺ T cells in the skin of mice sensitized with mosquito salivary gland extract is increased compared to that in the skin of non-sensitized mice (FIG. 4).

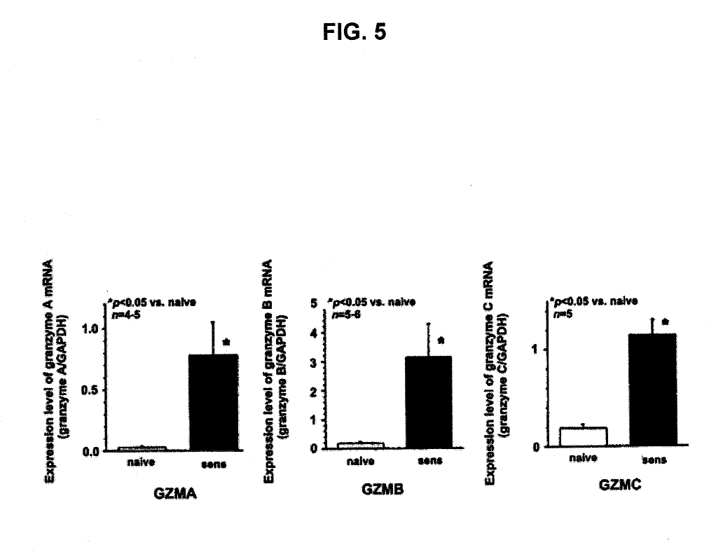

(5) Granzymes, which have been reported to be expressed in lymphocytes, were detected in real-time PCR for which subtype is expressed in the skin. Granzymes A, B, and C were increased in the skin of mice sensitized with mosquito salivary gland extract (FIG. 5).

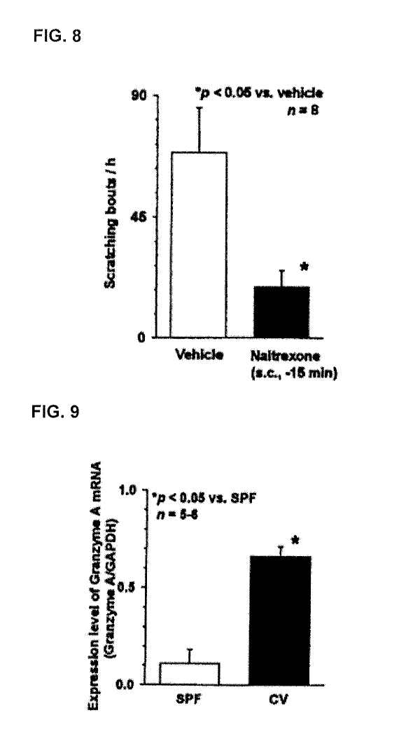

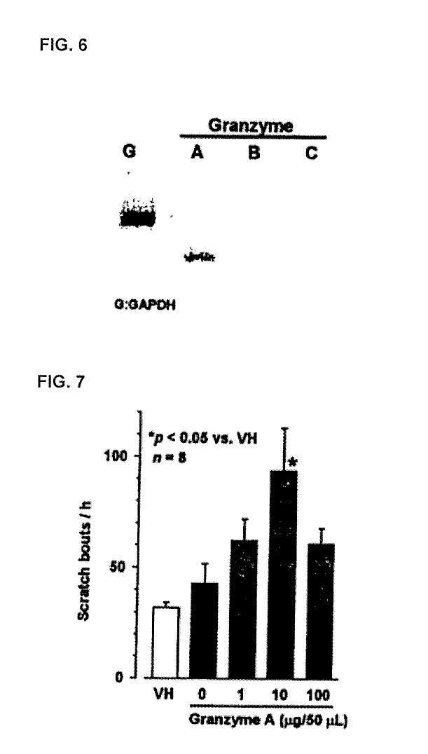

(6) In the CD4⁺ T cells isolated from the skin, only granzyme A was expressed (FIG. 6).

(7) Scratching behavior was induced in normal mice by being intradermally injected with granzyme A, having a peak at 10 μg/site (FIG. 7).

(8) Scratching behavior is induced by intradermal injection of granzyme A (FIG. 7), and the scratching behavior is suppressed by naltrexone (FIG. 8). This is supposed to be an itching reaction.

(9) In NC mice with atopic dermatitis, the expression of mRNA of granzyme A was increased in mice (conventional feeding) having itching or dermatitis, compared to that in mice (SPF feeding) not having itching and dermatitis (FIG. 9).

According to the present invention, it is possible to provide an index of intractable pruritic skin diseases on which conventional antiallergic agents are poorly effective, and it is thereby possible to easily and exactly diagnose such diseases and to develop a novel allergic disease therapeutic agent based on the action mechanism of granzyme A.

1. A biomarker for allergic disease, comprising a polynucleotide having a part of base sequence of granzyme A gene and/or a polynucleotide complementary thereto.

2. The biomarker according to claim 1, wherein the allergic disease is attributed to allergic reaction that is caused not exclusively by histamine release, and wherein the biomarker is used as a probe or a primer in testing for the disease.

3. The biomarker according to claim 1, wherein the allergic disease is accompanied by pruritus.

4. A method for diagnosing allergic disease, comprising the following steps (A) and (B):
(A) measuring the expression level of granzyme gene in a biological sample from a subject; and
(B) determining the presence or absence of the allergic disease based on the measurement result in step (A).

5. A method for diagnosing allergic disease, comprising the following steps (a), (b), and (c):
(a) binding the biomarker according to claim 1 to an RNA derived from a biological sample from a subject or a complementary polynucleotide transcripted from the RNA;
(b) measuring the RNA derived from the biological sample or the complementary polynucleotide transcripted from the RNA by the biomarker as an indication; and
(c) determining the presence or absence of the allergic disease based on the measurement result in step (b).

6. A biomarker for allergic disease, comprising an antibody that recognizes granzyme A.

7. The biomarker according to claim 6, wherein the allergic disease is attributed to allergic reaction that is caused not exclusively by histamine release, and wherein the biomarker is used as an antibody for detecting granzyme A in examination of the disease.

8. The biomarker according to claim 6, wherein the allergic disease is accompanied by pruritus.

9. A method for diagnosing allergic disease, comprising the following steps (A) and (B):
(A) measuring the expression level of granzyme A in a biological sample from a subject; and
(B) determining the presence or absence of the allergic disease based on the measurement result in step (A).

10. A method for diagnosing an allergic disease, comprising the following steps (a), (b), and (c):
(a) binding the biomarker according to claim 6 to protein prepared from a biological sample of a subject;
(b) measuring the protein derived from the biological sample by the bound biomarker as an indication; and
(c) determining the presence or absence of the allergic disease based on the measurement result in step (b).

11. A screening method of a substance capable of suppressing expression of granzyme A, comprising the following steps (a), (b), and (c):
(a) exposing a test substance to a cell that allows measurement of the expression of granzyme A;
(b) comparing with the expression level of granzyme A in a control cell which is not exposed to the test substances by measuring the expression level of granzyme A in the cell exposed to the test substance; and
(c) selecting a test substance that can decrease the expression level of granzyme A based on the comparison result in step (b).

12. A screening method of a substance capable of suppressing pruritus, the method including the following steps (a), (b), and (c):
(a) contacting a test substance with granzyme A;
(b) measuring inhibition of activity of granzyme A by the test substance; and
(c) selecting a test substance that can suppress pruritus based on the measurement result in step (b).

13. The screening method according to claim 12, wherein the pruritus is due to an allergic disease.

14. A therapeutic agent for allergic disease comprising an antibody that binds to granzyme A.

15. A therapeutic agent for allergic disease comprising a substance that suppresses the expression level of granzyme A as an effective ingredient.

16. The therapeutic agent according to claim 15, wherein the substance is an antibody against granzyme A or an expression vector containing a nucleic acid molecule encoding the antibody.

17. The therapeutic agent according to claim 15, wherein the agent has a serine protease-inhibiting activity.

18. An allergic disease therapeutic agent comprising a substance that suppresses expression of granzyme A gene as an effective ingredient.

19. The therapeutic agent according to claim 18, wherein the substance is an antisense nucleic acid, a ribozyme, a decoy nucleic acid, or a siRNA against granzyme A gene.

20. The therapeutic agent according to claim 18, wherein the agent has a serine protease-inhibiting activity.


SEQUENCE LISTING (10 sequences)

SEQ ID NO: 1 - protein, 262 aa, Homo sapiens
MRNSYRFLASSLSVVVSLLLIPEDVCEKIIGGNEVTPHSRPYMVLLSLDRKTICAGALIAKDWVLTAAHCNLNKRSQVILGAHSITREEPTKQIMLVKKEFPYPCYDPATREGDLKLLQLTEKAKINKYVTILHLPKKGDDVKPGTMCQVAGWGRTHNSASWSDTLREVNITIIDRKVCNDRNHYNFNPVIGMNMVCAGSLRGGRDSCNGDSGSPLLCEGVFRGVTSFGLENKCGDPRGPGVYILLSKKHLNWIIMTIKGAV

SEQ ID NO: 2 - DNA, 786 bp, Homo sapiens
atgaggaactcctatagatttctggcatcctctctctcagttgtcgtttctctcctgctaattcctgaagatgtctgtgaaaaaattattggaggaaatgaagtaactcctcattcaagaccctacatggtcctacttagtcttgacagaaaaaccatctgtgctggggctttgattgcaaaagactgggtgttgactgcagctcactgtaacttgaacaaaaggtcccaggtcattcttggggctcactcaataaccagggaagagccaacaaaacagataatgcttgttaagaaagagtttccctatccatgctatgacccagccacacgcgaaggtgaccttaaacttttacagctgacggaaaaagcaaaaattaacaaatatgtgactatccttcatctacctaaaaagggggatgatgtgaaaccaggaaccatgtgccaagttgcagggtgggggaggactcacaatagtgcatcttggtccgatactctgagagaagtcaatatcaccatcatagacagaaaagtctgcaatgatcgaaatcactataattttaaccctgtgattggaatgaatatggtttgtgctggaagcctccgaggtggaagagactcgtgcaatggagattctggaagccctttgttgtgcgagggtgttttccgaggggtcacttcctttggccttgaaaataaatgcggagaccctcgtgggcctggtgtctatattcttctctcaaagaaacacctcaactggataattatgactatcaagggagcagtt

SEQ ID NO: 3 - DNA, 20 bp, Artificial
  primer
tggaggagacacggttgttc

SEQ ID NO: 4 - DNA, 20 bp, Artificial
  primer
gagggagctgacttattgcc

SEQ ID NO: 5 - DNA, 20 bp, Artificial
  primer
cctacatggccttactttcg

SEQ ID NO: 6 - DNA, 20 bp, Artificial
  primer
aacctcttgtagcgtgtttg

SEQ ID NO: 7 - DNA, 20 bp, Artificial
  primer
agataatcggaggcaatgag

SEQ ID NO: 8 - DNA, 20 bp, Artificial
  primer
cacctgatccttctgtactg

SEQ ID NO: 9 - DNA, 20 bp, Artificial
  primer
ccaaggtcatccatgacaac

SEQ ID NO: 10 - DNA, 20 bp, Artificial
  primer
ttactccttggaggccacgt